(19) United States
(12) Patent Application Publication
(10) Pub. No.: US 2023/0355534 A1
(43) Pub. Date: Nov. 9, 2023

(54) METHOD TO ENCAPSULATE AN AQUEOUS SOLUTION COMPRISING A COMPOUND OF INTEREST IN A MATRIX

(71) Applicants: SECOYA TECHNOLOGIES, Louvain-la-Neuve (BE); UNIVERSITÉ LIBRE DE BRUXELLES, Bruxelles (BE)

(72) Inventors: Youen VITRY, ETTERBEEK (BE); Jean SEPTAVAUX, OTTIGNIES-LOUVAIN-LA-NEUVE (BE); Adrien DEWANDRE, LUSTIN (BE); Benoit SCHEID, LA HULPE (BE)

(21) Appl. No.: 18/246,005

(22) PCT Filed: Sep. 23, 2021
(86) PCT No.: PCT/EP2021/076177

§ 371 Date: Mar. 20, 2023

(30) Foreign Application Priority Data
Sep. 23, 2020 (EP) 20197680.0

(51) Int. Cl. A61K 9/50; A61K 38/42; B01J 13/18

(52) U.S. Cl. CPC A61K 9/5026; A61K 9/5089; A61K 9/5015; A61K 38/42; B01J 13/18

(57) ABSTRACT

A method for encapsulating a compound of interest in a matrix may include preparing a first aqueous solution including the compound of interest and a gelating agent. An oil or oil mixture and a second aqueous solution having a gelation inducing agent are prepared. The first aqueous solution is emulsified in the oil or oil mixture to form an emulsion of aqueous solution droplets in oil. That emulsion is emulsified in the second aqueous solution. The gelation inducing agent from the second aqueous solution is diffused through the oil towards the interface of the oil and the first aqueous solution. Gelation of the gelating agent at the interface forms the matrix, wherein particles are obtained including the matrix composed of the gelified gelating agent and encapsulating the compound of interest.

### TECHNICAL FIELD

The present disclosure is related to a method for encapsulating a compound of interest in a matrix. The compound of interest is in particular an active pharmaceutical ingredient (API) or a biomolecule, such as a protein.

### INTRODUCTION

The provision of one or more compounds of interest in a particle has gained much interest over the years, because it offers a lot of uses and applications in different domains, and in particular in biochemistry, bioscience and the medical domain. Particular compounds of interest for being contained or comprised in a particle are biomolecules, such as proteins, and/or active pharmaceutical ingredients (API). One of the applications of these compounds is the delayed or gradual disposal or delivery of these compounds. For example, the compounds can be released only in a certain environment, such as the acid environment of the human stomach. This makes it possible to release such compounds, such as pharmaceutical ingredients and drug, in a controlled way and at the optimal location to be the most effective.

Several methods for containing or the provision of compounds of interest in a particle exist today, such as coating (see for example “Mucoadhesive microparticulates based on polysaccharide for target dual drug delivery of 5-aminosalicylic acid and curcumin to inflamed colon”, Duan et al., Colloids Surf. B Biointerfaces, vol. 145, 2016, p. 510-519) or spray-drying (see for example “Pharmacokinetics of colon-specific pH and time-dependent flurbiprofen tablets”, Vemula et al., Eur. J. Drug Metab. Pharmacokinet. vol. 40, 2015, p. 301-311).

A method of particular interest is encapsulation, such as microencapsulation, wherein a compound of interest, such as an active agent, can be stored particle-wise within a shell which is surrounded by a matrix or a continuous film. The matrix or continuous film is typically composed of a polymeric material.

Water-in-oil-in-water double emulsions are disclosed in for example WO 2016/179251, wherein the double emulsion comprises an emulsifier and a surfactant to ensure its stability. Volatile chemical compounds, e.g. derivatives of cyclopropane, are encapsulated by this stable double emulsion.

US 2010/0233221 also discloses a water-in-oil-in-water double emulsion, wherein the stability of the water-in-oil emulsion and of the double emulsion is improved by using a minimum of two emulsifiers with varied molar mass.

WO 2017/199008 discloses a hydrogel-in-oil-in-water double emulsion containing emulsifiers. The inner aqueous phase comprises polymers that are subjected to crosslinking at elevated temperature.

WO2020036501 discloses a method for encapsulation of a lipophilic and hydrophilic compound based on a water-in-oil-in-water double emulsion that is stabilized with a hydrophobized derivative of hyaluronic acid.

The publications by Vinner et at. (“Microencapsulation of Clostridium difficile specific bacteriophages using microfluidic glass capillary devices for colon delivery using pH triggered release”, 2017, PLoS ONE 12) and by Vinner and Malik (“High precision microfluidic microencapsulation of bacteriophages for enteric delivery”, Res. Microbiol. Vol 169, 2018, p. 522-530) disclose a water-in-oil emulsion, wherein the oil phase comprises mygliol-840 oil and p-toluenesulfonic acid or 4-aminobenzoic acid. Droplets comprising the molecule of interest are obtained by means of gelation. The droplets are rinsed and washed with a solvent such as n-hexane.

The methods of the state of the art all have at least one or more of the following disadvantages:
additional components are needed to ensure a stable emulsion, such as one or more emulsifiers and/or a surfactant,
formation of the polymer film or shell by means of crosslinking at elevated temperatures or photo-initiated curing, which require the compounds of interest to be encapsulated to resist these conditions,
use of a solvent for obtaining the double emulsion, which requires the compounds of interest to be resistant to the solvent or to be kept away from the solvent, and wherein recycling or reuse of the solvent is often limited, rendering the method not environmentally friendly,
need for rinsing and washing of the obtained particles with a solvent, which is typically a toxic solvent such as n-hexane, which renders the process not environmentally friendly.

### SUMMARY

It is the aim of the present disclosure to provide a method for encapsulating a compound of interest, wherein the method of the present disclosure provides a solution to one or more of the problems encountered with the methods disclosed in the state of the art.

It is a particular aim of the present disclosure to provide a method for encapsulating a compound of interest, wherein the method does not use any toxic solvent. This reduces and may even exclude the need for cleaning the formed particles at the end of the production procedure, and makes the method more environmentally friendly.

The present disclosure further aims to provide a method for encapsulating a compound or compounds of interest, such as an active pharmaceutical ingredient (API) and/or a biomolecule, for example a protein, by means an emulsion, or a so-called double emulsion, of a first aqueous solution in oil or oil mixture in a second aqueous solution.

According to a first aspect of the present disclosure, the method for encapsulating a compound of interest in a matrix comprises the following steps:
preparing a first aqueous solution comprising the compound of interest and a gelating agent;
preparing an oil or oil mixture;
preparing a second aqueous solution comprising a gelation inducing agent;
emulsifying the first aqueous solution comprising the compound of interest and the gelating agent in the oil or oil mixture, wherein an emulsion of aqueous solution droplets in oil is formed;
emulsifying the aqueous solution droplets in oil in the second aqueous solution comprising the gelation inducing agent, wherein an emulsion of first aqueous solution in oil in second aqueous solution is formed;
diffusion of the gelation inducing agent from the second aqueous solution through the oil or oil mixture towards the interface of the oil or oil mixture and the first aqueous solution;
gelation of the gelating agent at the interface of the first aqueous solution and the oil by interaction between the diffused gelation inducing agent and the gelating agent, thereby forming a matrix, wherein particles are obtained comprising the matrix composed of the gelified gelating agent and encapsulating the compound of interest.

Advantageously, the gelation inducing agent is a crosslinker.

Advantageously, according to the method of the first aspect of the present disclosure:
the gelating agent is a polymer, preferably an ionic polymer when dissolved in the first aqueous solution;
the first aqueous solution has a pH higher or lower than a predetermined value;
the gelation inducing agent is a proton (H⁺) when the first aqueous solution has a pH higher than a predetermined value, and the gelation inducing agent is a hydroxide ion (OH⁻) when the first aqueous solution has a pH lower than a predetermined value;
the second aqueous solution has a pH lower or higher than the predetermined value when the first aqueous solution has a pH higher or lower than the predetermined value; and
protons, respectively hydroxide ions, diffuse from the second aqueous solution having a pH lower, respectively higher, than the predetermined value through the oil towards the interface of the oil and the first aqueous solution having a pH higher, respectively lower, than the predetermined value.

According to a second aspect of the present disclosure, the method for encapsulating a compound of interest in a matrix comprises the following steps:
preparing a first aqueous solution comprising the compound of interest and a polymer, preferably an ionic polymer when dissolved in the first aqueous solution, wherein the first aqueous solution has a pH higher or lower than a predetermined value;
preparing an oil or oil mixture;
preparing a second aqueous solution comprising a gelation inducing agent, wherein
   the second aqueous solution has a pH lower or higher than the predetermined value when the first aqueous solution has a pH higher or lower than the predetermined value; and
   the gelation inducing agent is a proton (H+) when the first aqueous solution has a pH higher than a predetermined value, and is a hydroxide ion (OH⁻) when the first aqueous solution has a pH lower than a predetermined value;
emulsifying the first aqueous solution comprising the compound of interest and the polymer in the oil or oil mixture, wherein an emulsion of aqueous solution droplets in oil is formed;
emulsifying the aqueous solution droplets in oil in the second aqueous solution comprising the gelation inducing agent, wherein an emulsion of first aqueous solution in oil in second aqueous solution is formed;
diffusion of the protons, respectively hydroxide ions, from the second aqueous solution having a pH lower, respectively higher, than the predetermined value through the oil towards the interface of the oil and the first aqueous solution having a pH higher, respectively lower, than the predetermined value;
gelation of the dissolved polymer at the interface of the first aqueous solution and the oil by interaction between the diffused protons or hydroxide ions and the polymer, thereby forming the matrix, wherein particles are obtained comprising the matrix composed of the gelified dissolved polymer and encapsulating the compound of interest.

According to a third aspect of the present disclosure, the method for encapsulating a compound of interest in a matrix comprises the following steps:
preparing a first aqueous solution comprising the compound of interest and a gelating molecule;
preparing an oil or oil mixture;
preparing a second aqueous solution comprising a gelation inducing molecule, preferably a crosslinking molecule;
emulsifying the first aqueous solution comprising the compound of interest and the gelating molecule in the oil or oil mixture, wherein an emulsion of aqueous solution droplets in oil is formed;
emulsifying the aqueous solution droplets in oil in the second aqueous solution comprising the gelation inducing molecule, wherein an emulsion of first aqueous solution in oil in second aqueous solution is formed;
diffusion of the gelation inducing molecule from the second aqueous solution through the oil or oil mixture towards the interface of the oil or oil mixture and the first aqueous solution;
gelation of the gelating molecule at the interface of the first aqueous solution and the oil by interaction between the diffused gelation inducing molecule and the gelating molecule, thereby forming the matrix, wherein particles are obtained comprising the matrix composed of the gelified gelating molecule and encapsulating the compound of interest.

According to a fourth aspect of the present disclosure, the method for encapsulating a compound of interest in a matrix comprises the following steps:
preparing a first aqueous solution comprising the compound of interest and a dissolved polymer;
preparing an oil or oil mixture;
preparing a second aqueous solution comprising a gelation inducing molecule, preferably a crosslinking molecule;
emulsifying the first aqueous solution comprising the compound of interest and the dissolved polymer in the oil or oil mixture, wherein an emulsion of aqueous solution droplets in oil is formed;
emulsifying the aqueous solution droplets in oil in the second aqueous solution comprising the gelation inducing molecule, wherein an emulsion of first aqueous solution in oil in second aqueous solution is formed;
diffusion of the gelation inducing molecule from the second aqueous solution through the oil or oil mixture towards the interface of the oil or oil mixture and the first aqueous solution;
gelation of the dissolved polymer at the interface of the first aqueous solution and the oil by interaction between the diffused gelation inducing molecule and the dissolved polymer, thereby forming the matrix, wherein particles are obtained comprising the matrix composed of the gelified dissolved polymer and encapsulating the compound of interest.

According to a fifth aspect of the present disclosure, the method for encapsulating a compound of interest in a matrix comprises the following steps:
preparing a first aqueous solution comprising the compound of interest and a gelating molecule;
preparing an oil or oil mixture;
preparing a second aqueous solution comprising an ion;
emulsifying the first aqueous solution comprising the compound of interest and the gelating molecule in the oil or oil mixture, wherein an emulsion of aqueous solution droplets in oil is formed;
emulsifying the aqueous solution droplets in oil in the second aqueous solution comprising the ion, wherein an emulsion of first aqueous solution in oil in second aqueous solution is formed;
diffusion of the ion from the second aqueous solution through the oil or oil mixture towards the interface of the oil or oil mixture and the first aqueous solution;
gelation of the gelating molecule at the interface of the first aqueous solution and the oil by interaction between the diffused ion and the gelating molecule, thereby forming the matrix, wherein particles are obtained comprising the matrix composed of the gelified gelating molecule and encapsulating the compound of interest.

According to a sixth aspect of the present disclosure, the method for encapsulating a compound of interest in a matrix comprises the following steps:
preparing a first aqueous solution comprising the compound of interest and a dissolved polymer;
preparing an oil or oil mixture;
preparing a second aqueous solution comprising an ion;
emulsifying the first aqueous solution comprising the compound of interest and the dissolved polymer in the oil or oil mixture, wherein an emulsion of aqueous solution droplets in oil is formed;
emulsifying the aqueous solution droplets in oil in the second aqueous solution comprising the ion, wherein an emulsion of first aqueous solution in oil in second aqueous solution is formed;
diffusion of the ion from the second aqueous solution through the oil or oil mixture towards the interface of the oil or oil mixture and the first aqueous solution;
gelation of the dissolved polymer at the interface of the first aqueous solution and the oil by interaction between the diffused ion and the dissolved polymer, thereby forming the matrix, wherein particles are obtained comprising the matrix composed of the gelified dissolved polymer and encapsulating the compound of interest.

The method according to any one of the aspects of the present disclosure may further comprise the step of separation of the particles from the oil or the oil mixture. The method may also further comprise the step of evaporation of the water encapsulated in the matrix composed of the gelified gelating agent.

When the gelating agent is a polymer, for example a dissolved polymer, the polymer advantageously is a copolymer. When the polymer is an ionic polymer when dissolved in the first aqueous solution, it is advantageously an ionic copolymer. The copolymer, preferably the ionic copolymer, is advantageously a (meth)acrylate copolymer. Preferably, the (meth)acrylate copolymer is a copolymer of an alkyl (meth)acrylate and (meth)acrylic acid, wherein the alkyl is a linear or branched C₁₋₄ alkyl. Preferably, the copolymer is a copolymer of an alkyl methacrylate and methacrylic acid. The (meth)acrylate copolymer is advantageously a copolymer of methyl methacrylate and methacrylic acid (C₁ alkyl) (poly(methacrylic acid-co-methyl methacrylate) or ethyl methacrylate and methacrylic acid (C₂ alkyl) (poly(methacrylic acid-co-ethyl methacrylate).

The oil may comprise oleic acid and/or one or more free fatty acids and/or one or more triglycerides, such as vegetable oil. Advantageously the oil or oil mixture comprises oleic acid.

The second aqueous solution advantageously comprises water and an acid. Preferably, the second aqueous solution comprises water and acetic acid.

The molecule of interest can be an active pharmaceutical ingredient (API). Alternatively, or additionally, the molecule of interest can be a biomolecule, such as a protein.

The method of the present disclosure may be performed by using a device such as the device disclosed in WO 2019/007965, hereby incorporated by reference. The device advantageously comprises:
an input capillary comprising two coaxial capillaries, wherein a first, inner, coaxial capillary comprises the first aqueous solution comprising the compound of interest and the gelating agent and a second, outer, coaxial capillary comprises the oil or oil mixture;
a cavity comprising the second aqueous solution comprising the gelation inducing agent; and
an output capillary coaxially aligned with the input capillary;
   wherein the opening of a tip of the input capillary has an internal diameter of smaller than the internal diameter of the output capillary, and wherein the cross-section of the cavity is selected so that, in use, the average speed field in the cavity is quasi-static.

The present disclosure further discloses a method using the above described device, wherein the method comprises the following steps:
injecting the first aqueous solution comprising the compound of interest and the gelating agent in the first, inner, coaxial capillary of the input capillary comprising two coaxial capillaries;
injecting the oil or oil mixture in the second, outer, coaxial capillary of the input capillary comprising two coaxial capillaries;
providing the second aqueous solution comprising the gelation inducing agent in a cavity;
emulsifying the first aqueous solution comprising the compound of interest and the gelating agent in the oil or oil mixture, wherein the emulsion of the aqueous solution droplets in oil in second aqueous solution is formed;
emulsifying the aqueous solution droplets in oil in the second aqueous solution, wherein the emulsion of the first aqueous solution in oil in second aqueous solution is formed;
collecting the emulsion of first aqueous solution in oil in second aqueous solution through the output capillary.

According to a further aspect of the present disclosure, the use of a method according to the one of the foregoing aspects is disclosed for the encapsulation of an active pharmaceutical ingredient (API) and/or a biomolecule. The encapsulation is advantageously an encapsulation in a matrix composed of the gelified gelating agent.

### BRIEF DESCRIPTION OF THE DRAWINGS

Aspects of the present disclosure will now be described in more detail with reference to the appended drawings, wherein same reference numerals illustrate same features and wherein:

FIG. 1 represents a schematic representation of the steps of a method of the present disclosure.

FIG. 2 represents a schematic representation of the particles being formed.

FIG. 3 represents a schematic representation of an alternative method of the present disclosure.

FIGS. 4 and 5 represent the production of a double emulsion of the present disclosure.

FIG. 6 represents a double emulsion obtained using a method of the present disclosure.

FIG. 7 represents a double emulsion obtained, wherein the double emulsion is located at the surface of the second aqueous solution.

FIG. 8 represents the obtained particles after gelation and separation from the oil or oil mixture.

FIG. 9 represents the obtained particles, wherein the particles have a mean diameter of 280 μm.

### DETAILED DESCRIPTION

In the light of the present disclosure, it is meant with “double emulsion” an emulsion of a first phase in a second phase in a third phase. More specifically, in the present disclosure, the double emulsion is an emulsion of a first aqueous solution in an oil or oil mixture in a second aqueous solution. The first aqueous solution is advantageously emulsified in the oil or oil mixture in the form of droplets.

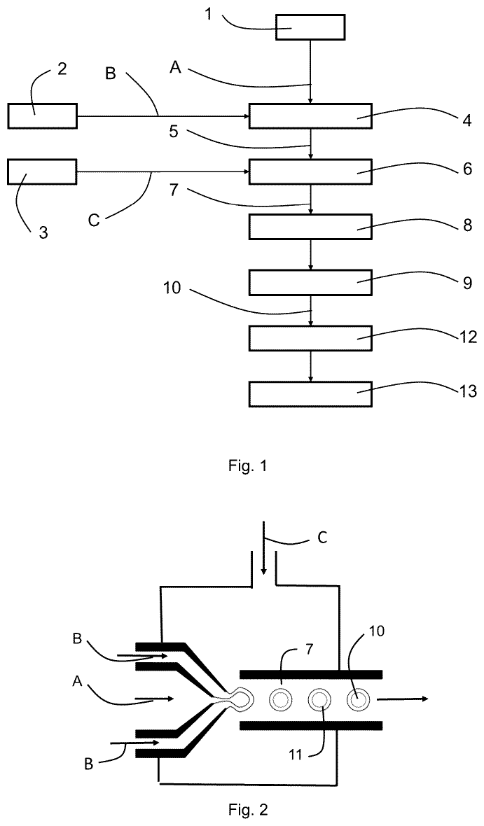

Referring to FIG. 1, a schematic representation of the steps of a method of the present disclosure is disclosed.

The method comprises a step of preparing (1) a first aqueous solution (A) comprising a compound of interest and a gelating agent, a step of preparing (2) an oil or oil mixture (B), and a step of preparing (3) a second aqueous solution (C) comprising a gelation inducing agent. The method further comprises the step of emulsifying (4) the first aqueous solution (A) comprising the compound of interest and the gelating agent in the oil or oil mixture (B), wherein an emulsion of aqueous solution droplets in oil (5; A in B) is formed. The emulsion of aqueous solution droplets in oil (5) is then emulsified (6) in the second aqueous solution (C) comprising the gelation inducing agent, wherein an emulsion of first aqueous solution in oil in second aqueous solution is formed (7; A in B in C). The emulsion of first aqueous solution in oil in second aqueous solution (7) is a double emulsion. When the double emulsion (7) is obtained, the gelation inducing agent diffuses (8) from the second aqueous solution (C) through the oil or oil mixture (B) towards the interface of the oil or oil mixture (B) and the first aqueous solution (A). Following the diffusion (8), the gelating agent gelates (9) at the interface of the first aqueous solution (A) and the oil (B) by interaction between the diffused gelation inducing agent and the gelating agent, thereby forming the matrix. During the gelation step (9), particles (10) are obtained comprising the matrix composed of the gelified gelating agent (11) and encapsulating the compound of interest.

The gelating agent can comprise one or more compounds, for example one or more components. The gelating agent is a gelating molecule, i.e. a molecule that gelates to form the matrix. The gelating agent can be a molecule such as polyamine. The gelating agent can be a monomer. The gelating agent can be a polymer, such as a copolymer. The polymer can be a neutral or an ionic polymer when dissolved in the first aqueous solution. The ionic polymer can be an anionic polymer or a cationic polymer. The gelating agent is at least partially dissolved in the first aqueous solution, such as at least 80% (mass based) of the gelating agent is dissolved, such as at least 85%, at least 90%, at least 95%, at least 98%, or at least 99%. Preferably, the gelating agent is entirely dissolved in the first aqueous solution (100% dissolved).

Preferably, the gelating agent is a (co)polymer, more preferably an ionic (co)polymer when dissolved in the first aqueous solution.

An anionic polymer is usually only soluble in an alkaline environment, i.e. an environment having a pH higher than a predetermined value. An anionic polymer is usually non-soluble, and will gelate or precipitate, in non-alkaline environments, such as an acidic environment, i.e. an environment having a pH lower than the predetermined value. For example, the predetermined value can be pH 7.

A cationic polymer is usually only soluble in an acidic environment, i.e. an environment having a pH lower than a predetermined value. A cationic polymer is usually non-soluble, and will gelate or precipitate, in non-acidic environments, such as an alkaline environment, i.e. an environment having a pH higher than the predetermined value. For example, the predetermined value can be pH 7.

The solubility and non-solubility of the gelating agent in relation to the environment wherein it is present, allows the encapsulation of the compound of interest, and maintained encapsulation of this compound in specific environments, for example an acidic environment, and release in other environments, for example a more alkaline environment.

Based on the environment where the encapsulated compound of interest must be released, and the environment(s) it has to resist, encapsulation based on a specific gelating agent, for example an anionic or cationic polymer, is preferred.

One particular example of interest is the delivery of an active pharmaceutical ingredient (API), such as a drug, in the human intestines. For release in the intestines, the API must pass the gastric environment, which is very acidic. Hence, encapsulation of the API in a polymer matrix obtained from an anionic polymer as gelating agent, such as an anionic copolymer, will ensure maintained encapsulation and thus no release of the API in the acidic gastric environment, and release in the intestines, which are a more alkaline environment.

Preferably, the ionic (co)polymer is a (meth)acrylate copolymer, such as a copolymer of an alkyl (meth)acrylate and (meth)acrylic acid, wherein the alkyl is a linear or branched C₁₋₂₀ alkyl, such as a C₁₋₁₄ alkyl, a C₁₋₁₂ alkyl, a C₁₋₁₀ alkyl, a C₁₋₈ alkyl, a C₁₋₆ alkyl, and preferably a C₁₋₄ alkyl, such as a C₂ alkyl (ethyl) or a C₁ alkyl (methyl). With a C₁₋₂₀ alkyl it is meant that the alkyl chain comprises between 1 and 20 carbon atoms, such as between 1 and 16, 14, 12, 10, 8, 6, 5, 4, or 3 carbon atoms.

The copolymer is advantageously a copolymer of an alkyl methacrylate and methacrylic acid. The (meth)acrylate copolymer is advantageously a copolymer of methyl methacrylate and methacrylic acid (C₁ alkyl), i.e. the copolymer poly(methacrylic acid-co-methyl methacrylate), or ethyl methacrylate and methacrylic acid (C₂ alkyl), i.e. the copolymer poly(methacrylic acid-co-ethyl methacrylate).

The gelating agent can be a polysaccharide. Example of suitable polysaccharides are, without being limited thereto, agarose, alginic acid, carboxymethyl cellulose, carrageenan, cellulose, cellulose acetate, cellulose acetate propionate, chitosan, cyclodextrins, dextran, ethyl cellulose, hyaluronic acid, hydroxypropyl methyl cellulose, hydroxypropyl methylcellulose, starch, gum tragacanth, zein, pectin, carboxymethyl cellulose, phtalic acid acetic acid cellulose, methyl cellulose, hypromellose, carageenan, xyloglucan, or curdlan.

Alternatively, the gelating agent can be a polyacrylate. Examples of suitable polyacrylates are, without being limited thereto, poly(hydroethylmethacrylate), polyethylvinylacetate, poly(3-sulfopropyl methacrylate potassium salt), poly(4-vinylbenzoic acid), poly(acrylic acid), poly(ethylacrylic acid), poly(ethyleneglycol acrylate phosphate), poly(ethyleneglycol methacrylate phosphate), poly(itaconic acid), poly(methylmethacrylate), poly(N-ethylpyrrolidine methacrylate), poly(propylacrylic acid), poly[(2-diethylamino)ethylmethacrylate], poly[(2-diisopropylamino)ethylmethacrylate], poly[(2-dimethylamino)ethylacrylate], poly[(2-dimethylamino)ethylmethacrylate], poly[(2-dipropylamino)ethylmethacrylate], poly[(2-N-morpholino)ethylmethacrylate], poly[2-(tert-butylamino)ethylmethacrylate], poly[6-(1H-imidazol-1-yl)hexylmethacrylate], or polymethacrylate.

Alternatively, the gelating agent can be a polyester. Examples of suitable polyesters are, without being limited thereto, poly(dioxanones), poly(hydroxy butyrate), poly(β-malic acid), poly(ε-caprolactone), poly-glycolic acid, poly-lactic acid, poly-lactic glycolic acid, poly(caprolactone), or poly(valerolactone).

Alternatively, the gelating agent can be a polyacrylamide. Examples of suitable polyacrylamides are, without being limited thereto, poly(2-acrylamido-2-methylpropane sulfonic acid), poly(3-acrylamidophenyl boronic acid), poly[(2-diethylamino)ethyl acrylamide], poly[(2-N-morpholino)ethyl methacrylamide], or poly[N-(3-(dimethylamino)-propyl) methacrylamide].

Alternatively, the gelating agent can be one of the following: polyamides, polyphosphates, polyphosphonates, polyamines, polyamino acids such as pPoly(aspartic acid), poly(histidine), poly(L-glutamic acid), and Poly(lysine); poly(2-vinylpyridine), poly(4-styrenesulfonic acid), poly(4-vinyl-benzyl phosphonic acid), poly(4-vinylpyridine), poly(acryloylmorpholine), poly(amidoamine), poly(ethylenimine), poly(N,N-dialkylvinylbenzylamine), poly(N-acryloyl-N′-alkenyl piperazine), poly(N-vinylimidazole), poly(propylenimine), poly(vinylphenyl boronic acid), poly(vinylphophonic acid), or poly(vinylsulfonic acid).

Alternatively, the gelating agent can be a protein. Examples of suitable proteins are, without being limited thereto, albumin, casein, collagen, gelatin, globulin, proteoglycan, or elastin.

Alternatively, the gelating agent can be a polyanhydride. Examples of preferred polyanhydrides are, without being limited thereto, poly(adipic acid), poly(sebacic acid), or poly(terephtalic acid).

The compound of interest comprised in the first aqueous solution can be an active pharmaceutical ingredient (API). Alternatively, or additionally, the compound of interest can be a biomolecule or a combination of biomolecules. The biomolecule can be a protein. Examples of preferred proteins are haemoglobin, such as pork haemoglobin, and lysozyme.

The oil or oil mixture advantageously comprises a fatty acid or a combination of two or more fatty acids. Examples of preferred fatty acids are, without being limited thereto, oleic acid, linoleic acid, and palmitoleic acid. A preferred fatty acid is oleic acid, hence the oil or oil mixture advantageously comprises oleic acid. The fatty acid can be a free fatty acid. Alternatively or additionally, the oil or oil mixture can comprise one or more triglycerides, such as vegetable oil. Examples of vegetable oil are linseed oil, olive oil, coconut oil, soybean oil, rapeseed oil, sunflower oil, cottonseed oil, castor oil, peanut oil, macadamia oil, cashew oil, walnut oil, sesame oil, and corn oil. The oil or oil mixture can be biocompatible.

The gelation inducing agent is advantageously a crosslinking agent. The gelation inducing agent can be a molecule, for example CO₂, or an ion, for example Ca²⁺ or a hydroxide ion (OH⁻), or can be a proton (H⁺).

The type of gelation inducing agent that is comprised in the second aqueous solution depends, without being limited thereto, on the composition of the first aqueous solution, in particular of the gelating agent and the compound of interest. The gelation inducing agent must be capable of diffusing through the oil or oil mixture, preferably without the gelation inducing agent being degraded, damaged, altered or reacted, and must also be capable to interact with the gelating agent (gelation) to obtain the matrix, without interacting (e.g. reacting) with or degrading the compound of interest to be comprised within the obtained matrix.

The interaction between the diffused gelation inducing agent and the gelating agent can be a chemical reaction, such as a reaction forming covalent bonds, for example a polymerization reaction or a crosslinking reaction. The interaction can be an interaction wherein hydrogen bonds are formed. The interaction can be one interaction or a combination of interactions, such as a polymerization and crosslinking reaction, wherein a monomer is polymerized and a polymeric, crosslinked network is obtained (i.e. the matrix is a polymeric, crosslinked network).

When the gelation inducing agent is a proton (H⁺), the second aqueous solution can comprise water and one or more acids. When the gelation inducing agent is a hydroxide ion (OH⁻), the second aqueous solution can comprise water and one or more bases. When the gelation inducing agent is an ion, in particular a Ca²⁺ ion, the second aqueous solution can comprise water and one or more calcium salts, such as, without being limited thereto, calcium carbonate (CaCO₃), calcium chloride (CaCl₂)), calcium iodide (CaI₂), calcium nitrite (Ca(NO₂)₂), calcium nitrate hydrate (Ca(NO₃)₂·xH₂O), calcium oxalate (CaC₂O₄), or calcium sulphate (CaSO₄). When the gelation inducing agent is a molecule, in particular CO₂, the second aqueous solution can comprise water and dissolved CO₂.

When the second aqueous solution comprises one or more acids, the acid is preferably acetic acid. The acid allows for the provision of protons that can diffuse through the oil or oil mixture towards the interface of the oil or oil mixture and the first aqueous solution.

The second aqueous solution can optionally comprise one or more additives, such as a surfactant, a buffer, a salt, or a reagent.

The first aqueous solution can optionally comprise one or more additives, such as an acidity regulator, for example an acid or an alkaline molecule.

Preferably, the particles obtained with the method of the present disclosure have a mean diameter between 5 μm and 750 μm, such as between 10 μm and 500 μm, and preferably between 20 μm and 300 μm. The optimal mean diameter of the particles depends on the use of the particles.

Particles for use in oral administration typically have a mean diameter between 50 μm and 150 μm, preferably between 75 μm and 125 μm, more preferably between 90 μm and 110 μm, such as around 100 μm.

Particles for parenteral administration typically have a mean diameter between 5 μm and 50 μm, preferably between 5 μm and 30 μm, more preferably between 10 μm and 25 μm.

Referring back to FIG. 1, the method further comprises the optional step of separation (10) of the particles (8) from the oil or the oil mixture. The separation may be performed by means of centrifugation or spinning, by filtration, or by any other suited separation technique known in the art, or by a combination of two or more techniques.

The method represented in FIG. 1 further comprises the optional step of evaporation (11) of the water encapsulated in the matrix composed of the gelified gelating agent (9). The evaporation may be performed by heating the particles, for example to a temperature up to the softening or dissolution temperature of the gelified gelating agent. Preferably, the particles are heated under reduced pressure to temperatures between room temperature and 75° C., such as between 25° C. and 50° C.

Preferably, the gelating agent is a polymer, more preferably an ionic (co)polymer when dissolved in the first aqueous solution, i.e. an anionic (co)polymer or a cationic (co)polymer.

In the case of the gelating agent being an anionic (co)polymer, the first aqueous solution has a pH higher than the predetermined value, the second aqueous solution has a pH lower than the predetermined value and the gelation inducing agent is a proton (H⁺). The difference in pH between the first and the second aqueous solution allow the protons to diffuse from the second aqueous solution through the oil towards the interface of the oil and the first aqueous solution. Upon diffusion of the protons the pH of the first aqueous solution is reduced. When the pH of the first aqueous solution reaches a value lower than the predetermined value, the dissolved anionic (co)polymer is no longer soluble in the first aqueous solution and gelates at the interface of the first aqueous solution and the oil by interaction with the protons. A matrix is formed, wherein particles are obtained comprising the matrix composed of the gelified (co)polymer and encapsulating the compound of interest. The interaction of the (co)polymer with the protons is advantageously a polymerization reaction and/or a crosslinking reaction, wherein the gelified (co)polymer is advantageously a crosslinked polymeric network.

In the case of the gelating agent being a cationic (co)polymer, the first aqueous solution has a pH lower than the predetermined value, the second aqueous solution has a pH higher than the predetermined value and the gelation inducing agent is a hydroxide ion (OH⁻). The difference in pH between the first and the second aqueous solution allow the hydroxide ions to diffuse from the second aqueous solution through the oil towards the interface of the oil and the first aqueous solution. Upon diffusion of the hydroxide ions, the pH of the first aqueous solution is increased. When the pH of the first aqueous solution reaches a value higher than the predetermined value, the dissolved cationic (co)polymer is no longer soluble in the first aqueous solution and gelates at the interface of the first aqueous solution and the oil by interaction with the hydroxide ions. A matrix is formed, wherein particles are obtained comprising the matrix composed of the gelified (co)polymer and encapsulating the compound of interest. The interaction of the (co)polymer with the hydroxide ions is advantageously a polymerization reaction and/or a crosslinking reaction, wherein the gelified (co)polymer is advantageously a crosslinked polymeric network.

When the environment where release of the compound of interest is to be avoided is acidic, the gelating agent advantageously is an anionic (co)polymer that gelates or precipitates in an acidic environment and is soluble in a first aqueous solution having a pH higher than a predetermined value (i.e. alkaline first aqueous solution). Alternatively, when the environment where release of the molecule of interest is to be avoided is alkaline, the gelating agent advantageously is a cationic (co)polymer that gelates or precipitates in an alkaline environment and is soluble in a first aqueous solution having a pH lower than a predetermined value (i.e. acidic first aqueous solution).

Without being bound to any theory, the ionic polymer in the matrix may be present as a neutralized ionic polymer or as an ionic polymer. Preferably, the ionic polymer is present in the polymer matrix as a neutralized ionic polymer.

The predetermined value for the pH may be a value between 1 and 13, such as between 4 and 10, preferably between 6 and 8, such as 7. The pH value may be measured by means of a pH meter, preferably calibrated using reference solutions having a known pH value.

FIG. 2 represents schematically the method for encapsulation of a compound of interest in a matrix according to FIG. 1, wherein a double emulsion (7; A in B in C) of first aqueous solution (A) in oil or oil mixture (B) in second aqueous solution (C) is obtained by emsulsifying the first aqueous solution (A) comprising the compound of interest and the gelating agent in the oil or oil mixture (B), thereby forming an emulsion of aqueous solution droplets in oil, followed by emulsifying the aqueous solution droplets in oil in the second aqueous solution (C) comprising the gelation inducing agent. Upon diffusion of the gelation inducing agent from the second aqueous solution (C) through the oil or oil mixture (B) towards the interface of the oil or oil mixture (B) and the first aqueous solution (A), gelation of the gelating agent takes place by interaction between the diffused gelation inducing agent and the gelating agent. A matrix is thereby formed, wherein particles (10) are obtained comprising the matrix composed of the gelified gelating agent (11) and encapsulating the compound of interest.

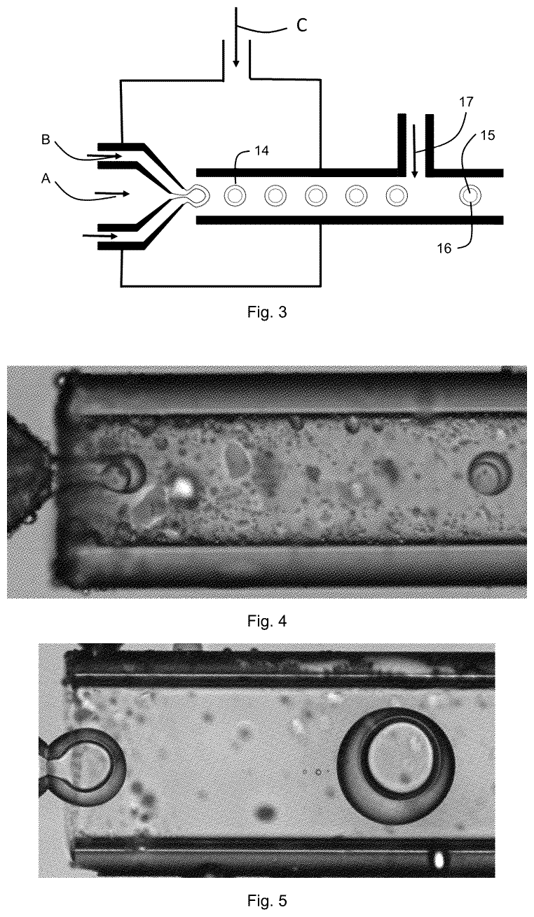

Referring to FIG. 3, an alternative method to the method of FIG. 2 is shown, comprising preparing a first aqueous solution (A), an oil or oil mixture (B) and a second aqueous solution (C). The first aqueous solution (A), the oil or oil mixture (B) and the second aqueous solution (C) are as described above for the method represented in FIG. 1 and FIG. 2. A double emulsion (14) of first aqueous solution (A) in oil (B) in second aqueous solution (C) is obtained by emsulsifying the first aqueous solution (A) comprising the compound of interest and the gelating agent in the oil or oil mixture (B), thereby forming an emulsion of aqueous solution droplets in oil, followed by emulsifying the aqueous solution droplets in oil in the second aqueous solution (C) comprising the gelation inducing agent.

The method of FIG. 3 further comprises a diffusion step wherein the gelation inducing agent diffuses through the oil or oil mixture (B) towards the interface of the first aqueous solution and the oil or oil mixture. The gelating agent gelates at the interface of the first aqueous solution (A) and the oil (B) by interaction between the diffused gelation inducing agent and the gelating agent, thereby forming a matrix composed of the gelified gelating agent (16). Particles (15) are obtained comprising the matrix composed of the gelified gelating agent (16) and encapsulating the compound of interest

The diffusion of the gelation inducing agent and the gelation can be delayed or accelerated by adding gelation inducing agent to the double emulsion (14) through an additional phase (17) having similar properties as the second aqueous solution. For example, in the particular case where the gelating inducing agent is respectively a proton or a hydroxide ion, the additional phase (17) will have a pH respectively lower or higher than a predetermined value.

Alternatively to the method shown in FIG. 3, the diffusion step and gelation step may also be delayed or accelerated by collecting the double emulsion in a reservoir comprising a solution having specific properties.

The solution is advantageously an acidic solution, such as a solution comprising an acid, such as acetic acid, when diffusion of protons is required, i.e. when the pH of the first aqueous solution is higher and the pH of the second aqueous solution is lower than a predetermined value.

The solution is advantageously an alkaline solution when diffusion of hydroxide ions is required, i.e. when the pH of the first aqueous solution is lower and the pH of the second aqueous solution is higher than a predetermined value. Examples of a suitable alkaline solution are sodium hydroxide (NaOH) and potassium hydroxide (KOH).

The method of the present disclosure may be performed by using a device advantageously comprising an input capillary comprising two coaxial capillaries, a cavity and an output capillary. Alternatively, the device may comprise two input capillaries, a cavity and an output capillary.

When the device comprises an input capillary comprising two coaxial capillaries, a first, inner, coaxial capillary advantageously comprises the first aqueous solution comprising the compound of interest and the gelating agent. A second, outer, coaxial capillary advantageously comprises the oil or oil mixture. The cavity advantageously comprises the second aqueous solution comprising the gelation inducing agent. The output capillary is advantageously coaxially aligned with the input capillary.

The opening of a tip of the input capillary may have an internal diameter smaller than the internal diameter of the output capillary. The cross-section of the cavity can be selected so that, in use, the average speed field in the cavity is quasi-static. The internal diameter of the opening of a tip of the input capillary can be up to 95% of the internal diameter of the output capillary, such as between 5% and 95%, for example between 10% and 90%, between 20% and 80%, between 25% and 75%, between 30% and 70%, such as around 50%.

The method using the above described device comprises the injection of a first aqueous solution comprising the compound of interest and the gelating agent in the first, inner, coaxial capillary of the input capillary comprising two coaxial capillaries, the injection of the oil or oil mixture in the second, outer, coaxial capillary of the input capillary comprising two coaxial capillaries, and providing the second aqueous solution comprising the gelation inducing agent in the cavity. The method further comprises the step of emulsifying the first aqueous solution comprising the compound of interest and the gelating agent in the oil or oil mixture, wherein the emulsion of the aqueous solution droplets in oil is formed, and the step of emulsifying the aqueous solution droplets in oil in the second aqueous solution, wherein the emulsion of the first aqueous solution in oil in second aqueous solution is formed (so-called double emulsion). The method further comprises the collection of the double emulsion through the output capillary.

According to a further aspect of the present disclosure, the use of a method according to the present disclosure is disclosed for the encapsulation of an active pharmaceutical ingredient (API) and/or a biomolecule. The encapsulation is advantageously an encapsulation in a matrix composed of gelified gelating agent.

### EXAMPLES

The following example demonstrates, without being limited thereto, methods for encapsulation of a compound of interest in a matrix according to the present disclosure.

Depending on the particle size that is required for the use of the particles produced by the method of the present disclosure, the parameters and settings of the device can be varied. Preferably, the inner diameter of the input capillary or capillaries and/or the inner diameter of the output capillary are varied, as well as the flow rate of the first and second aqueous solution and the oil or oil mixture. When the device comprises an input capillary comprising two coaxial capillaries (a so-called double nozzle), the inner diameter of the first, inner, coaxial capillary (first nozzle) and the diameter of the second, outer, coaxial capillary (second nozzle) can be varied. This double nozzle is introduced in a cavity comprising a continuous phase surrounding the liquid at the tip of the double nozzle, and the different phases are collected in an output tube located in front of the double nozzle in the cavity.

FIG. 4 shows a double emulsion (first aqueous solution in oil in second aqueous solution) obtained by a device comprising a double nozzle wherein the first nozzle has an inner diameter of 30 μm and the second nozzle has an inner diameter of 60 μm. The device further comprises an output capillary having an inner diameter of 180 μm. The mean diameter of the double emulsion obtained is 60 μm.

### Example 1

FIG. 5 shows a double emulsion (first aqueous solution in oil in second aqueous solution) obtained by a device comprising a double nozzle wherein the first nozzle has an inner diameter of 90 μm and the second nozzle has an inner diameter of 160 μm. The device further comprises an output capillary having an inner diameter of 450 μm. The first aqueous solution consisted of water, with 5 wt % Eudragit™ S100 (Methacrylic acid-methylmetacrylate copolymer) as the gelating agent, 0.5 wt % of pork haemoglobin as the compound of interest. This first aqueous solution was injected in the inner nozzle at a flow rate of 17.3 μl/min. The second fluid was oleic acid, and was introduced in the second nozzle at a flow rate of 11 μl/min. Finally, the continuous phase forming the second aqueous phase, introduced in the cavity at a flow rate of 780 μl/min consisted of water, 1 wt % acetic acid and 1% PVA. The mean diameter of the double emulsion obtained is 300 μm at the output of the output capillary.

The double emulsion obtained (first aqueous solution in oil or oil mixture in second aqueous solution) was then collected from the device. FIG. 6 shows a collected double emulsion in solution. The double emulsion is monodisperse. The core of each double emulsion is in this specific example composed of a dissolved methacrylate copolymer and pork haemoglobin, which is a protein, as molecule of interest. The obtained emulsion is left some minutes at rest to let the gelating agent diffuse into the core solution to induce gelation.

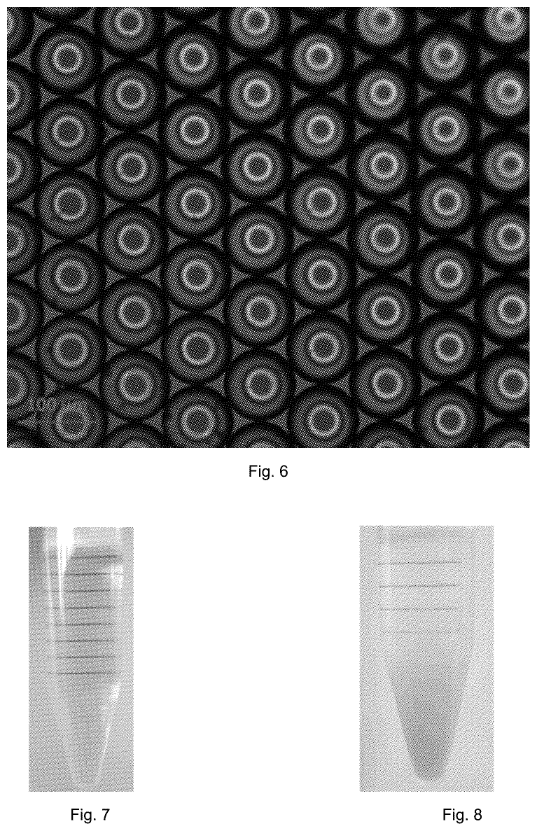

FIG. 7 shows another collected solution, also called a bulk solution, wherein the double emulsion is located at the surface.

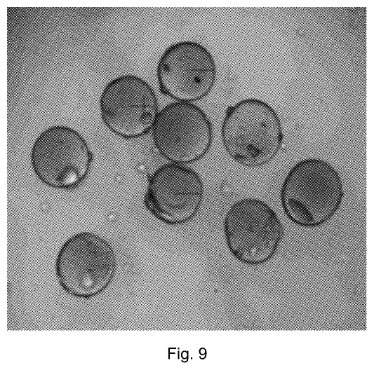

Following a diffusion step of the gelating inducing agent from the second aqueous solution through the oil phase to the first aqueous solution and a gelation step according to the methods of the present disclosure, the obtained particles were separated from the solution. FIG. 8 shows the particles after separation from the solution by centrifugation. FIG. 9 shows a detail of the particles of FIG. 8, indicating a mean diameter of 280 μm having a deviation of only 3%. All the experiment was carried out at room temperature.

The encapsulation efficiency has been measured through spectrophotometry measurement by means of a Thermoscientific Genesys 180 device. The encapsulation efficiency is measured by measuring the percentage of compound of interest in the second aqueous solution after separation of the obtained particles. The percentage is a weight percentage, i.e. the mass of compound in relation to the total mass of the second aqueous solution. Given the high mass of second aqueous solution in relation to the mass of the compound of interest, only significant amounts of compound of interest are detected. A typical detection limit is 2 w %. If no compound of interest is detected in the second aqueous solution after separation of the obtained particles, it means that no to very low quantities of compound of interest are present in the second aqueous solution, and that the encapsulation can be considered to have been efficient. Detection of the compound of interest indicates less efficient encapsulation. The larger the amount of compound of interest detected, the less efficient was the encapsulation.

For the measurement of the encapsulation efficiency, the protein pork haemoglobin was used as compound of interest for encapsulation. No traces of the protein pork haemoglobin were detected, indicating that almost all up to all the protein was encapsulated in the particles and that the encapsulation was performed efficiently.

A second evaluation method measures the encapsulation yield. The encapsulation yield is measured directly on the particles, and represents the percentage of the compound of interest initially used in the encapsulation method that is detected in the particles. The measurement is performed by spectrophotometry by means of a Thermoscientific Genesys 180 device after dissolution of the particles. Based on the detection limits of the device, the upper limit of encapsulation yield is noticed to be 98%, meaning that approximately 2% is not detected. This also means that an encapsulation yield of 98% indicates a very good encapsulation of the compound of interest.

For the measurement of the encapsulation yield, the protein pork haemoglobin was used as compound of interest for encapsulation. The encapsulation yield for pork haemoglobin was up to 98%, indicating a very high encapsulation yield.

### Example 2

The same conditions and device as in example 1 were used. The first aqueous solution (core) was composed of water with 2 wt % of alginate as gelating agent, the oil mixture (shell) is composed of Soybean oil with 1 wt % of Abil 90 (emulsifier) and the second aqueous solution (continuous phase) is composed of water with 2 wt % of calcium chloride as gelation inducing agent and 1 wt % of Tween 20 (Polyoxyethylene (20) sorbitan monolaurate). The three phases are injected at room temperature with the respective flowrates of 1.1, 1.3 and 149 μL/min at room temperature. Particles of 100 μm are obtained after gelation.

### Example 3

The same conditions and device as in example 1 were used. The first aqueous solution (core) was composed of water with 2 wt % of chitosan (30-100 cps) as gelating agent, the oil mixture (shell) is composed of Soybean oil with 1 wt % of Abil 90 and the second aqueous solution (continuous phase) is composed of water with 1 wt % of Glutaraldehyde as gelation inducing agent and 1 wt % of Tween 20. The three phases are injected at room temperature with the respective flowrates of 3.6, 4.6 and 126 μL/min. Particles of 100 μm are obtained after gelation.

1. A method for encapsulating a compound of interest in a matrix, the method comprising the following steps:
preparing a first aqueous solution comprising the compound of interest and a gelating agent;
preparing an oil or oil mixture;
preparing a second aqueous solution comprising a gelation inducing agent;
emulsifying the first aqueous solution comprising the compound of interest and the gelating agent in the oil or oil mixture, wherein an emulsion of aqueous solution droplets in oil is formed;
emulsifying the aqueous solution droplets in oil in the second aqueous solution comprising the gelation inducing agent, wherein an emulsion of first aqueous solution in oil in second aqueous solution is formed;
diffusing the gelation inducing agent from the second aqueous solution through the oil or oil mixture towards the interface of the oil or oil mixture and the first aqueous solution;
forming a matrix by gelation of the gelating agent at the interface of the first aqueous solution and the oil by interaction between the diffused gelation inducing agent and the gelating agent, wherein particles are obtained comprising the matrix including the gelified gelating agent and encapsulating the compound of interest.

2. The method according to claim 1, wherein the gelation inducing agent is a crosslinker.

3. The method according to claim 1, wherein
the gelating agent is a polymer;
the gelation inducing agent is a proton (H⁺) when the first aqueous solution has a pH higher than a predetermined value;
the second aqueous solution has a pH lower than the predetermined value when the first aqueous solution has a pH higher than the predetermined value; and
protons diffuse from the second aqueous solution having a pH lower than the predetermined value through the oil towards the interface of the oil and the first aqueous solution having a pH higher than the predetermined value.

4. The method according to claim 1, further comprising the step of separating the particles from the oil or oil mixture.

5. The method according to claim 1, further comprising the step of evaporation of the water encapsulated in the matrix composed of the gelified gelating agent.

6. The method according to claim 3, wherein the polymer is a acrylate copolymer.

7. The method according to claim 6, wherein the acrylate copolymer is a copolymer of an alkyl acrylate and acrylic acid, wherein the alkyl is a linear or branched C₁₋₄ alkyl.

8. The method according to claim 6, wherein the acrylate copolymer is a copolymer of methyl methacrylate and methacrylic acid or ethyl methacrylate and methacrylic acid.

9. The method according to claim 1, wherein the oil comprises oleic acid.

10. The method according to claim 1, wherein the oil comprises one or more free fatty acids.

11. The method according to claim 1, wherein the second aqueous solution comprises water and acetic acid.

12. The method according to claim 1, wherein the compound of interest is an active pharmaceutical ingredient (API).

13. The method according to claim 1, wherein the compound of interest is a biomolecule.

14. The method according to claim 1, wherein a device for generating droplets is used, the device comprising:
an input capillary comprising two coaxial capillaries, wherein a first, inner, coaxial capillary comprises the first aqueous solution comprising the compound of interest and the gelating agent and a second, outer, coaxial capillary comprises the oil or oil mixture;
a cavity comprising the second aqueous solution comprising the gelation inducing agent; and
an output capillary coaxially aligned with the input capillary;
wherein the opening of a tip of the input capillary has an internal diameter smaller than the internal diameter of the output capillary, and wherein the cross-section of the cavity is configured so that, in use, the average speed field in the cavity is quasi-static.

15. The method according to claim 14, wherein the method comprises the following steps:
injecting the first aqueous solution comprising the compound of interest and the gelating agent in the first, inner, coaxial capillary of the input capillary comprising two coaxial capillaries;
injecting the oil or oil mixture in the second, outer, coaxial capillary of the input capillary comprising two coaxial capillaries;
providing the second aqueous solution comprising the gelation inducing agent in a cavity;
emulsifying the first aqueous solution comprising the compound of interest and the gelating agent in the oil or oil mixture, wherein the emulsion of aqueous solution droplets in oil is formed;
emulsifying the aqueous solution droplets in oil in the second aqueous solution, wherein the emulsion of the first aqueous solution in oil in second aqueous solution is formed;
collecting the emulsion of first aqueous solution in oil in second aqueous solution through the output capillary.

16. The method according to claim 1, wherein
the gelating agent is a polymer when dissolved in the first aqueous solution;
the gelation inducing agent is a hydroxide ion (OH⁻) when the first aqueous solution has a pH lower than a predetermined value;
the second aqueous solution has a pH higher than the predetermined value when the first aqueous solution has a pH lower than the predetermined value; and
hydroxide ions diffuse from the second aqueous solution having a pH higher than the predetermined value through the oil towards the interface of the oil and the first aqueous solution having a pH lower than the predetermined value.

17. The method of claim 3, wherein the gelating agent is an ionic polymer when dissolved in the first aqueous solution.

18. The method of claim 7 wherein the (meth)acrylate copolymer is a copolymer of an alkyl methacrylate and methacrylic acid.

19. The method of claim 13, wherein the compound of interest is a protein.
